(19) United States
(12) Patent Application Publication
(10) Pub. No.: US 2025/0361225 A1
(43) Pub. Date: Nov. 27, 2025

(54) A PROCESS FOR PREPARATION OF 3-[5-(DIFLUOROMETHOXY)-1- METHYL-3-(TRIFLUOROMETHYL)PYRAZOL-4- YLMETHYLSULFONYL]-4,5-DIHYDRO-5,5-DIMETHYL-1,2-OXAZOLE AND ITS INTERMEDIATES

(71) Applicant: UPL LIMITED, Mumbai (IN)

(72) Inventors: Prashant Vasant KINI, Mumbai (IN); Sopan Nagnath GANDHALE, Mumbai (IN); Debasish SENGUPTA, Mumbai (IN); Sandip Sahebrao GULVE, Mumbai (IN); Shrikant Muqutrao ANPAT, Mumbai (IN)

(21) Appl. No.: 18/854,116

(22) PCT Filed: Apr. 6, 2023
(86) PCT No.: PCT/IB2023/053549

§ 371 Date: Oct. 4, 2024

(30) Foreign Application Priority Data
Apr. 8, 2022 (IN) 202221021181

(51) Int. Cl. C07D 413/12

(52) U.S. Cl. CPC C07D 413/12

(57) ABSTRACT

The present invention relates to a process for preparation of 3-[5-(difluoromethoxy)-1-methyl-3-(trifluoromethyl)pyrazo 1-4-ylmethylsulfonyl]-4,5-dihydro-5,5-dimethyl-1,2-oxazole and its intermediates. The present invention provides a process for preparation of 3-({[5-fluoro-1-methyl-3-(trifluoromethyl)-1H-pyrazol-4-yl]methyl}sulfanyl)-5,5-dimethyl-4,5-dihydro-1,2-oxazole and 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole in the absence of N,N-dimethyl formamide.

### FIELD OF THE INVENTION

The present invention relates to a process for preparation of 3-[5-(difluoromethoxy)-1-methyl-3-(trifluoromethyl)pyrazol-4-ylmethylsulfonyl]-4,5-dihydro-5,5-dimethyl-1,2-oxazole and its intermediates.

### BACKGROUND OF THE INVENTION

Pyroxasulfone is a selective pre-emergence grass and broadleaved weed herbicide. It is a pre-emergence herbicide that inhibits the biosynthesis of very long chain fatty acids. It can be used effectively to control grass and broad-leaved weeds in corn, soybean and wheat fields. It belongs to the class of isoxazoline herbicide. The International Union of Pure and Applied Chemistry (IUPAC) name for pyroxasulfone is 3-[5-(difluoromethoxy)-1-methyl-3-(trifluoromethyl)pyrazol-4-ylmethylsulfonyl]-4,5-dihydro-5,5-dimethyl-1,2-oxazole and is represented by the compound of formula (I).

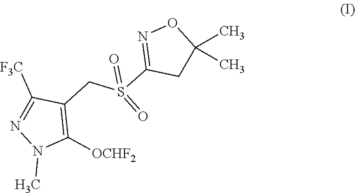 [SMILES: Cn1nc(C(F)(F)F)c(CS(=O)(=O)C2=NOC(C)(C)C2)c1OC(F)F]

U.S. Pat. No. 7,256,298 discloses a process for preparation of pyroxasulfone wherein the compound of formula (IIa) is prepared by reaction of 3-ethanesulfonyl-5,5-dimethyl-2-isoxazoline compound of formula (IVa) with 2-(5-difluoromethoxy-1-methyl-3-trifluoromethyl-1H-pyrazole-4-ylmethyl)-isothiourea hydrobromide compound of formula (IIIc) using anhydrous potassium carbonate base in the presence of ethanol, water and N,N-dimethyl formamide (DMF) solvent. Compound of formula (IIa) was then subjected to oxidation to obtain pyroxasulfone of formula (I). The reaction scheme can be represented as in Scheme I.

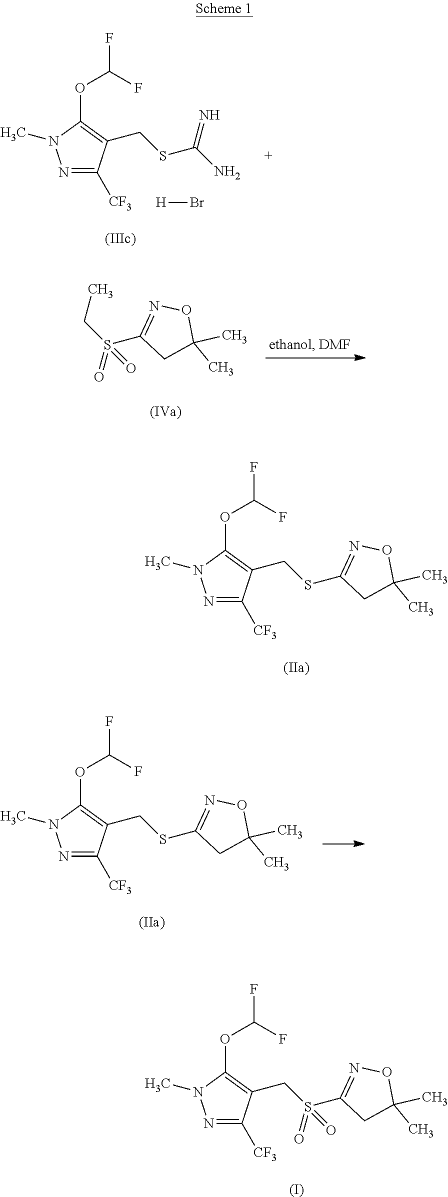

It was observed by the inventors of present invention, that when the afore-mentioned process was followed to prepare a compound analogous to formula (IIa), undesired by-products of structural formula (V) were also formed. Compounds of formula (V) wherein R¹ is selected from hydrogen, halogen, C₁ to C₁₀ alkyl group, C₃ to C₈ cycloalkyl group, C₃ to C₈ cycloalkyl group, C₁ to C₃ alkyl group or haloalkyl group were difficult to remove/separate from compound of formula (IIa) due to their structural similarity and were carried forward along with compound of formula (IIa) in further reactions, which impacted the purity and yield of the final product, i.e., pyroxasulfone.

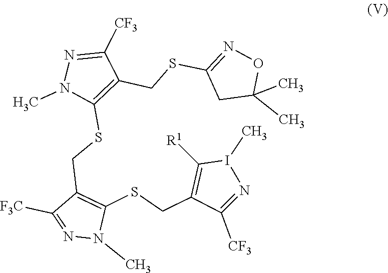

Another problem associated with the above prior art was the use of multiple solvent system containing ethanol, water and N,N-dimethyl formamide (DMF) for reaction. The recovery of two organic solvents i.e., ethanol and DMF from the DMF-water-ethanol solvent system was quite difficult at the industrial scale. Further, studies have also demonstrated a myriad of health hazards posed by DMF, primarily related to hepatotoxicity. Therefore, it is important to avoid use of such solvents on large scale production for the safety of the workers. Therefore, there is an unmet need for the development of an economical, eco-friendly, high yielding process for the preparation of high purity pyroxasulfone and its intermediates.

### OBJECTIVES OF THE INVENTION

It is a primary objective of the present invention to provide a compound of formula (II) substantially free from impurities.

It is another objective of the present invention to provide a process for preparation of the compound of formula (II) having high yield and purity.

It is yet another objective of the present invention to provide a simple, economical and industrially viable process for preparation of the compound of formula (II).

It is yet another objective of the present invention to provide pyroxasulfone of formula (I) substantially free from impurities.

It is yet another objective of the present invention to provide a process for preparation of pyroxasulfone of formula (I), substantially free from impurities.

### SUMMARY OF THE INVENTION

According to an aspect, the present invention provides a process for the preparation of a compound of formula (II), the process comprising:
condensing a compound of formula (III) or salts thereof with a compound of formula (IV),

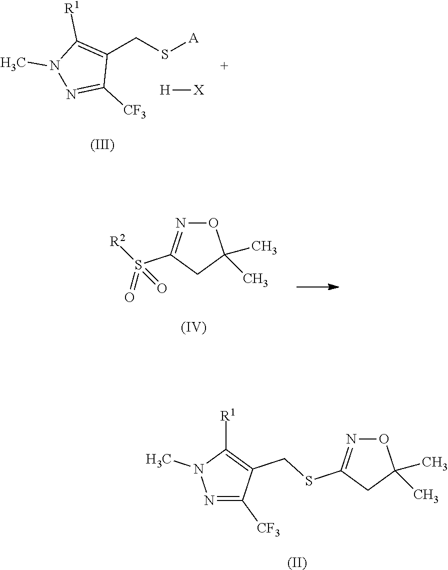

wherein X is halogen;
R² is C₁ to C₁₀ alkyl group;
A is selected from hydrogen, —C(NH)(NH₂) or alkali metal; and
R¹ is selected from hydrogen, halogen, C₁ to C₁₀ alkyl group, C₃ to C₈ cycloalkyl group, C₃ to C₈ cycloalkyl group, C₁ to C₃ alkyl group or haloalkyl group; and wherein the reaction is carried out in the absence of N,N-dimethyl formamide.

According to another aspect, the present invention provides a compound of formula (II) substantially free from a compound of formula (V);

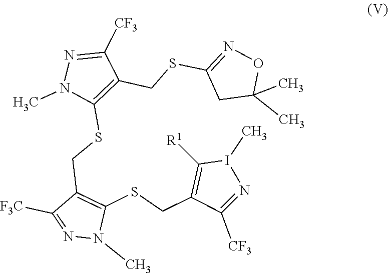

wherein R¹ is selected from hydrogen, halogen, C₁ to C₁₀ alkyl group, C₃ to C₈ cycloalkyl group, C₃ to C₈ cycloalkyl group, C₁ to C₃ alkyl group or haloalkyl group.

According to another aspect of present invention, there is provided a process for preparation of pyroxasulfone of formula (I), the process comprising:
a) preparing a compound of formula (IIa) by condensing a compound of formula (IIIa) or salts thereof with a compound of formula (IV) in the presence of a base; and wherein reaction is carried out in absence of N, N-dimethyl formamide;

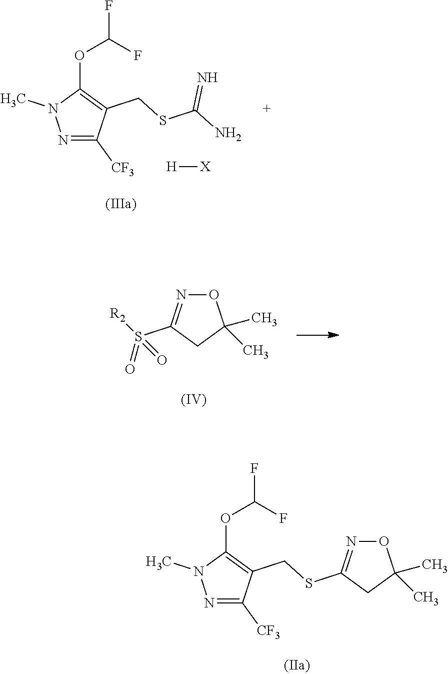

wherein X is halogen and R² is C₁ to C₁₀ alkyl group; and
b) oxidizing the compound of formula (IIa) to obtain pyroxasulfone of formula (I).

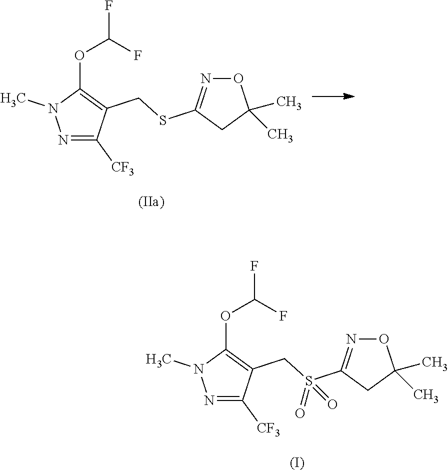

According to another aspect of present invention, there is provided a process for preparation of pyroxasulfone of formula (I), the process comprising:
a) preparing a compound of formula (IIa) by condensing a compound of formula (IIIa) or salts thereof with a compound of formula (IV) in the presence of a weak base; and wherein reaction is carried out in absence of N, N-dimethyl formamide;

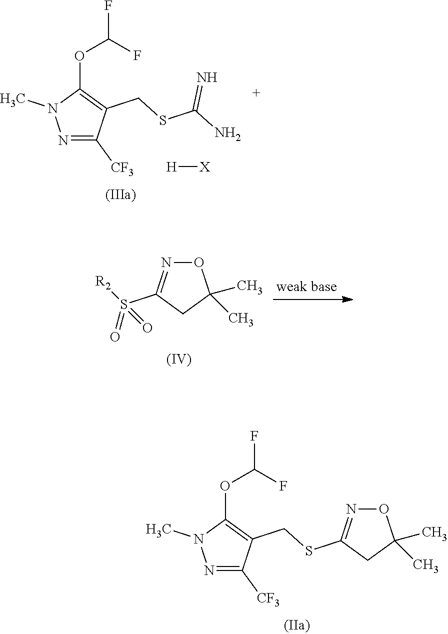

wherein X is halogen and R² is C₁ to C₁₀ alkyl group; and
b) oxidizing the compound of formula (IIa) to obtain pyroxasulfone of formula (I).

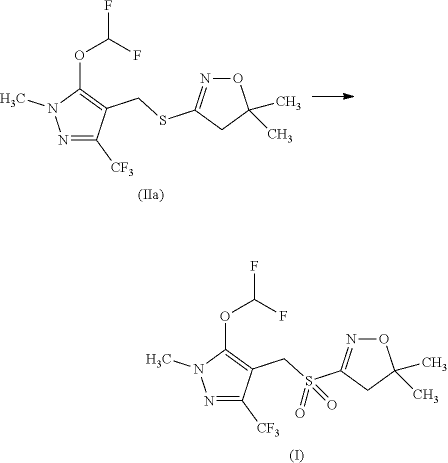

According to another aspect of present invention, there is provided a process for preparation of pyroxasulfone of formula (I), the process comprising:
a) preparing a compound of formula (IIb) by condensing a compound of formula (IIIb) or salts thereof with a compound of formula (IV), wherein the reaction is carried out in the absence of N, N-dimethyl formamide;

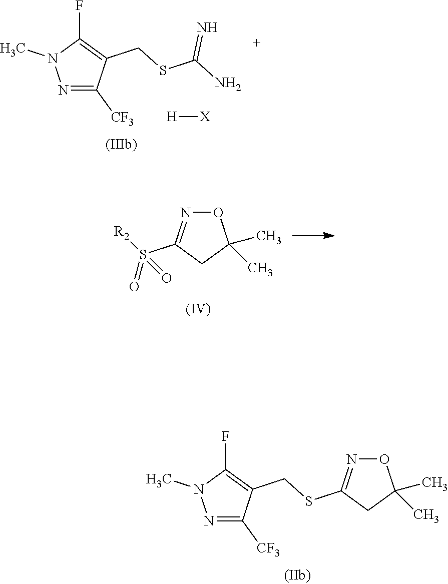

wherein X is halogen and R² is C₁ to C₁₀ alkyl group;
b) converting the compound of formula (IIb) to a compound of formula (IIa); and

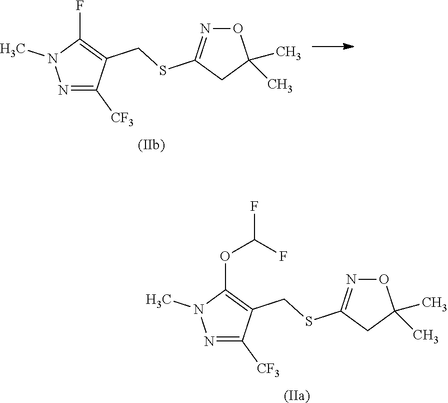

c) oxidizing the compound of formula (IIa) to obtain pyroxasulfone of formula (I).

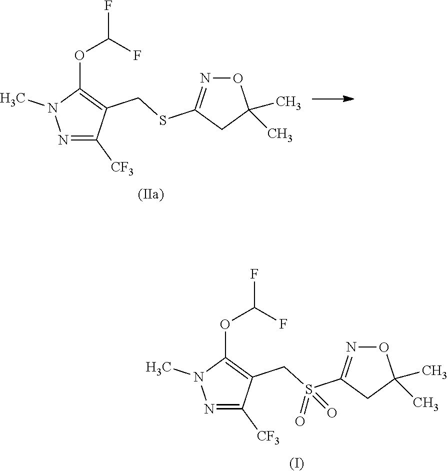

According to another aspect of present invention, there is provided pyroxasulfone of formula (I) substantially free from a compound of formula (V), wherein R¹ is selected from hydrogen, halogen, C₁ to C₁₀ alkyl group, C₃ to C₈ cycloalkyl group, C₃ to C₈ cycloalkyl group, C₁ to C₃ alkyl group or haloalkyl group.

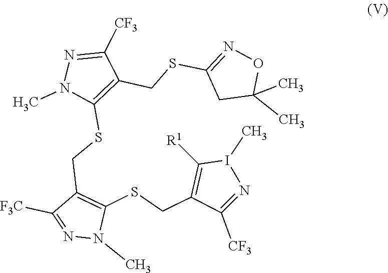

According to an aspect of present invention, there is provided a process for preparation of a compound of formula (IIb), the process comprising: condensing a compound of formula (IIIb) or salts thereof with a compound of formula (IV), wherein the reaction is carried out in absence of N, N-dimethyl formamide; and

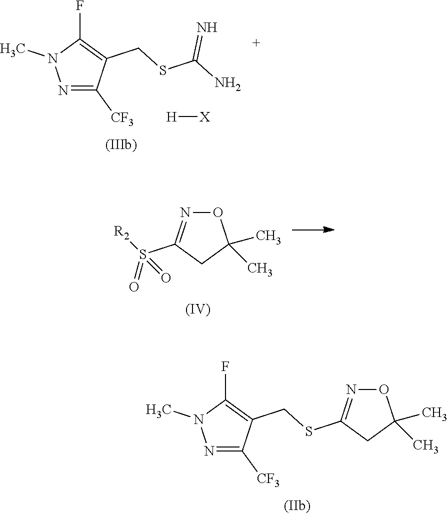

wherein X is halogen and R² is C₁ to C₁₀ alkyl group.

### BRIEF DESCRIPTION OF THE DRAWINGS

FIG. 1 illustrates a HPLC chromatogram of Example 1: 3-({[5-fluoro-1-methyl-3-(trifluoromethyl)-1H-pyrazol-4-yl]methyl}sulfanyl)-5,5-dimethyl-4,5-dihydro-1,2-oxazole.

FIG. 2 illustrates a HPLC chromatogram of Example 4: 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole.

FIG. 3 illustrates a HPLC chromatogram of Example 5: 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole.

FIG. 4 illustrates a HPLC chromatogram of Example 6: 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole.

FIG. 5 illustrates a HPLC chromatogram of Example 8: 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole.

FIG. 6 illustrates a HPLC chromatogram of Example 9: 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole.

FIG. 7 illustrates a HPLC chromatogram of Example 13: Pyroxasulfone.

FIG. 8 illustrates a HPLC chromatogram of comparative example 14: 3-({[5-fluoro-1-methyl-3-(trifluoromethyl)-1H-pyrazol-4-yl]methyl}sulfanyl)-5,5-dimethyl-4,5-dihydro-1,2-oxazole.

FIG. 9 illustrates a HPLC chromatogram of comparative example 15: 3-({[5-fluoro-1-methyl-3-(trifluoromethyl)-1H-pyrazol-4-yl]methyl}sulfanyl)-5,5-dimethyl-4,5-dihydro-1,2-oxazole.

### DETAILED DESCRIPTION OF THE INVENTION

Those skilled in art will be aware that invention described herein is subject to variations and modifications other than those specifically described. It is to be understood that the invention described herein includes all such variations and modifications. The invention also includes all such steps, features, compositions and methods referred to or indicated in this specification, individually or collectively, and any and all combinations of any two or more said steps or features.

For convenience, before further description of the present invention, certain terms employed in the specification, examples are described here. These definitions should be read in light of the remainder of the disclosure and understood as by a person of ordinary skill in the art. Unless defined otherwise, all technical and scientific terms used herein have the same meaning as commonly understood by a person of ordinary skill in the art. The terms used throughout this specification are defined as follows, unless otherwise limited in specific instances.

The terms used herein are defined as follows.

As used in the specification and the claims, the singular forms “a”, “an”, and “the” include plural referents unless the context clearly dictates otherwise.

The present disclosure is not to be limited in scope by the specific embodiments described herein, which are intended for the purposes of exemplification only.

The term “about” shall be interpreted to mean “approximately” or “reasonably close to” and any statistically insignificant variations therefrom.

As used herein, the terms “comprising”, “including”, “having”, “containing”, “involving”, and the like are to be understood to be open-ended, i.e., to mean including but not limited to.

The terms “preferred” and “preferably” refer to embodiments of the invention that may afford certain benefits, under certain circumstances. In an embodiment, the aspects and embodiments described herein shall also be interpreted to replace the clause “comprising” with either “consisting of” or with “consisting essentially of” or with “consisting substantially of”.

As used herein, the term “(C₁-C₁₀) alkyl” or “alkyl” alone or as part of a substituent group, refers to the radical of saturated aliphatic groups, including straight or branched-chain alkyl groups. A straight-chain or branched chain alkyl has six or more carbon atoms in its backbone, for instance, C₁-C₁₀ for straight chain. As used herein, (C₁-C₁₀) alkyl refers to an alkyl group having from 1 to 10 carbon atoms.

Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl, and 3-methylbutyl.

As used herein, the term “(C₃-C₈)-cycloalkyl” or “cycloalkyl”, whether used alone or as part of a substituent group, refers to a saturated cyclic hydrocarbon radical including 1, 2 or 3 rings including a total of 3 to 8 carbon atoms forming the rings, which can be unsubstituted or substituted with one or more substituents. The term cycloalkyl includes bridged, fused and spiro ring systems. As used herein, the “(C₃-C₈)-cycloalkyl” refers to a cycloalkyl group having 3 to 8 (both inclusive) carbon atoms. Representative examples of cycloalkyl include, but are not limited to, cyclopropyl, cyclobutyl, cyclopentyl, cyclohexyl and the like.

As used herein, the term “halo(C₁-C₃)alkyl” or “haloalkyl”, whether used alone or as part of a substituent group, refers to the alkyl group which is substituted with one or more halogens. A monohalo(C₁-C₃)alkyl radical, for example, can have a chlorine, bromine, iodine or fluorine atom. Dihalo- or polyhalo(C₁-C₃)alkyl radicals can have two or more of the same or different halogen atoms. Representative examples of halo(C₁-C₃)alkyl include, but are not limited to, chloromethyl, dichloromethyl, trichloromethyl, dichloroethyl, dichloropropyl, fluoromethyl, difluoromethyl, trifluoromethyl, pentafluoroethyl, heptafluoropropyl, difluorochloromethyl, dichlorofluoromethyl, difluoroethyl, difluoropropyl or the like groups.

The term “alkali metal” as used herein includes lithium, sodium, potassium, rubidium, and caesium and also alloys of two or more such metals with each other, for example, potassium sodium alloys.

As used herein, the term “halogen” or “halo” refers to a fluorine, chlorine, bromine, or iodine atom.

The term “room temperature” unless stated otherwise, essentially means temperature in range from about 20° C. to about 35° C.

The term “purity” means purity as determined by High Pressure Liquid Chromatography (HPLC) method.

As used herein, the term “a/a” means area/area which is the percentage area corresponding to the retention time peaks in the chromatograms disclosed in FIGS. 1-9.

As used herein in this context, the expression “substantially free” will be understood to mean that pyroxasulfone and/or the compound of formula (II) contains 5% or less, 2% or less, or 1% or less, or 0.5% or less of any known or unknown impurity, particularly compound of formula (V) as measured for example by the HPLC method.

As used herein in this context, the term “known or unknown impurity” refers to unreacted synthetic intermediates, reagents, solvents, organic and/or inorganic products of side reactions, organic and/or inorganic salts and/or other undesired materials. Therefore, the compounds of the invention being substantially free from impurities are intended to mean the referred compound being substantially free from the unreacted synthetic intermediates, reagents, solvents, organic and/or inorganic products of side reactions, organic and/or inorganic salts and/or other undesired materials.

The term “pyroxasulfone” or “compound of formula (I)” as used herein, includes pyroxasulfone free base or its salts or its crystalline forms and polymorphs and is used interchangeably throughout the disclosure.

As used herein, the term “agrochemical” refers to all active substances which can be used in the agrochemical field, i.e., fungicides, bactericides, insecticides, acaricides, nematicides, molluscicides, rodenticides, repellents, plant growth regulators, herbicides and safeners, and plant nutrients. Subsequently, the term “herbicides” encompasses both herbicides as well as plant growth regulators, in so far as this does not otherwise emerge from the context.

As used herein the term “herbicide” denotes a compound which controls or modifies the growth of undesired weeds/plants.

The term “control” relates to a weed, includes control of the weed, as well as protecting a plant, a portion of the plant, or a plant seed from attack or invasion by said weed.

The expression of various quantities in terms of “%” or “% (by weight)” or “% w/w” means the percentage by weight of the total composition unless otherwise specified.

The present disclosure is not to be limited in scope by the specific embodiments described wherein, which are intended for the purposes of exemplification only.

It has been noted that compound of formula (I) has very low solubility in water and in other hydrophilic solvents due to which isolation and purification of the compound is troublesome. The known synthetic routes result in multiple side products having uneven physical properties such as solubility, which require various purification methods and are not effective for isolating compound of formula (I) with high yield. Despite the low solubility in water and other hydrophilic solvents, it has been found by the inventors of the present invention that substantially pure compound of formula (I) can be prepared by carrying out the reaction in the absence of certain specific reagents.

According to an embodiment, there is provided a process for preparation of a compound of formula (II), the process comprising:
condensing a compound of formula (III) or salts thereof with a compound of formula (IV);
wherein reaction is carried out in absence of N,N-dimethyl formamide; and

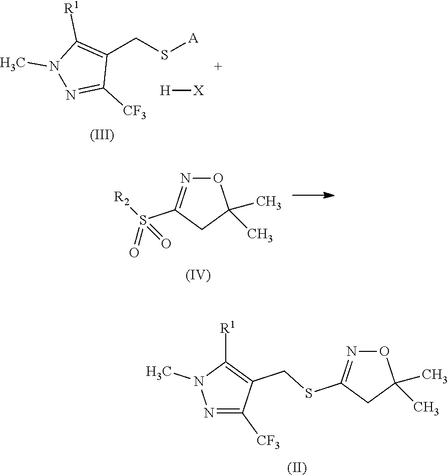

wherein X is halogen;
R² is C₁ to C₁₀ alkyl group;
A is selected from hydrogen, —C(NH)(NH₂) or alkali metal; and
R¹ is selected from hydrogen, halogen, C₁ to C₁₀ alkyl group, C₃ to C₈ cycloalkyl group, C₃ to C₈ cycloalkyl group, C₁ to C₃ alkyl group or haloalkyl group.

In another embodiment, the compounds of formula (III) and (IV) are prepared by any methods known in the prior art.

According to an embodiment, the process for preparation of the compound of formula (II) is carried out in presence of a base.

In another embodiment, the base used is selected from, but not limited to, an organic base or an inorganic base.

In another embodiment, the base used is selected from an organic base or an inorganic base.

According to an embodiment, the base used is an inorganic base.

In another embodiment, the inorganic base used is selected from, but not limited to, sodium hydroxide, potassium hydroxide, potassium carbonate, sodium carbonate, potassium bicarbonate, sodium bicarbonate, or combinations thereof.

According to an embodiment, the base used is an organic base.

In another embodiment, the organic base used is selected from, but not limited to, triethylamine, trimethylamine, pyridine, triethylene diamine, N,N-dimethyl pyridine, or combinations thereof.

In a preferred embodiment, the base is selected from the group comprising sodium hydroxide, potassium hydroxide, potassium carbonate, sodium carbonate, potassium bicarbonate, sodium bicarbonate, triethylamine, trimethylamine, pyridine, triethylene diamine, N,N-dimethyl pyridine, or combinations thereof.

According to an embodiment, for preparation of the compound of formula (II) the base used is an organic base selected from one or more of triethylamine, trimethylamine, pyridine, triethylene diamine, N,N-dimethyl pyridine, or combinations thereof.

According to an embodiment, the process for preparation of the compound of formula (II) is carried out in presence of a solvent.

In another embodiment, the solvent used is selected from, but not limited to alcohol, ketone, ester, ether, nitrile, aromatic or aliphatic hydrocarbon, halogenated aromatic or aliphatic hydrocarbon, water, or mixtures thereof.

In another embodiment, the solvent is selected from the group comprising alcohol selected from methanol, ethanol, isopropyl alcohol; ketone selected from acetone, methyl ethyl ketone; ester selected from methyl acetate, ethyl acetate; ether selected from isopropyl ether, petroleum ether, tetrahydrofuran; nitrile selected from acetonitrile; aromatic or aliphatic hydrocarbon selected from benzene, toluene, xylene, hexane; halogenated aromatic or aliphatic hydrocarbon selected from dichloromethane, dichloroethane, chloroform, carbon tetrachloride, chlorobenzene; water, or mixtures thereof.

According to an embodiment, the solvent used is selected from the group comprising methanol, ethanol, isopropyl alcohol, acetone, methyl ethyl ketone, methyl acetate, ethyl acetate, isopropyl ether, petroleum ether, tetrahydrofuran, acetonitrile, benzene, toluene, xylene, dichloromethane, dichloroethane, chloroform, carbon tetrachloride, chlorobenzene, water, or mixtures thereof.

According to an embodiment, the solvent used is selected from the group comprising water, ethanol, ethyl acetate, acetone, or mixtures thereof.

In a preferred embodiment, the process is carried out in presence of a solvent selected from methanol or ethanol.

According to an embodiment, the process for preparation of the compound of formula (II) is carried out in presence of a base and a solvent.

According to an embodiment, the process for preparation of the compound of formula (II) is carried out in presence of an inorganic base and a solvent.

According to an embodiment, the base used is potassium carbonate and solvent used is ethanol.

According to an embodiment, the base used is potassium carbonate and solvent used is ethyl acetate.

According to an embodiment, the base used is potassium carbonate and solvent used is acetone.

According to an embodiment, the base used is sodium carbonate and solvent used is ethanol.

According to an embodiment, the process for preparation of the compound of formula (II) is carried out in presence of an organic base and a solvent.

According to an embodiment, the base used is triethylamine and solvent used is ethanol.

According to an embodiment, the base used is trimethylamine and solvent used is ethanol.

According to an embodiment, the base used is pyridine and solvent used is ethanol.

In a preferred embodiment, the process for preparation of a compound of formula (IIb) comprises:
condensing a compound of formula (IIIb) or salts thereof with a compound of formula (IV);
wherein reaction is carried out in absence of N,N-dimethyl formamide; and
wherein X is halogen and R² is C₁ to C₁₀ alkyl group.

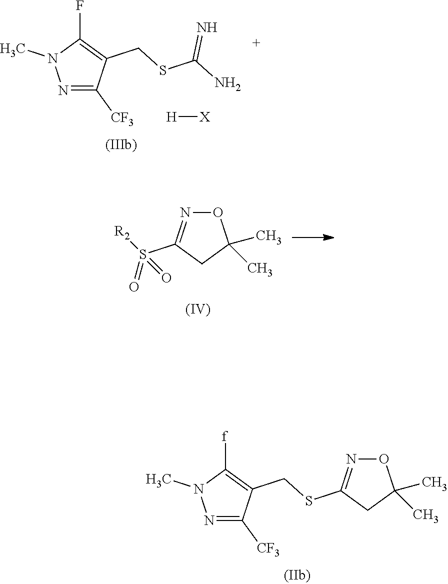

According to an embodiment, in the process for preparation of the compound of formula (II), the reaction is carried out at temperature ranging from about 0° C. to about 150° C.

In another embodiment, in the process for preparation of the compound of formula (II), the reaction is carried out at temperature ranging from about 10° C. to about 100° C.

In another embodiment, in the process for preparation of the compound of formula (II), the reaction is carried out at temperature ranging from about 20° C. to about 40° C.

According to an embodiment, the present invention provides a compound of formula (II) substantially free from impurities.

According to an embodiment, the impurities comprise unreacted synthetic intermediates, reagents, solvents, organic and/or inorganic products of side reactions, organic and/or inorganic salts, and/or other undesired materials.

According to an embodiment, the present invention provides the compound of formula (II) substantially free from unreacted synthetic intermediates, reagents, solvents, organic and/or inorganic products of side reactions, organic and/or inorganic salts, and/or other undesired materials.

In another embodiment, the following table discloses a list of the probable organic and/or inorganic products of side reactions, and/or other undesired materials.

| | Undesired products of side |
|---|---|
| | reactions in the compound of |
| Markush Structure | formula (II) |
| 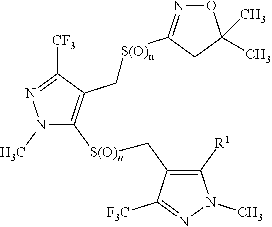 | 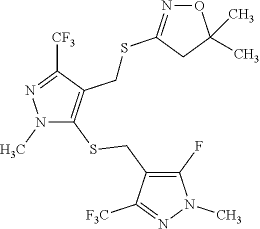 |
| (VI′) | (VIa′) |
| | 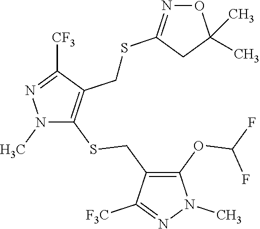 [SMILES: Cn1nc(C(F)(F)F)c(CSc2c(CSC3=NOC(C)(C)C3)c(C(F)(F)F)nn2C)c1OC(F)F] |
| | (VIb′) |
| | 1. n = 0, R¹ is F |
| | 2. n = 0, R¹ is OCHF₂ |
| 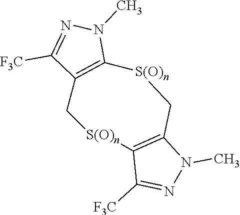 | 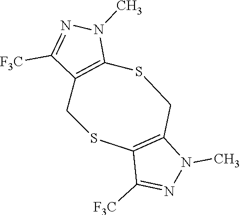 [SMILES: Cn1nc(C(F)(F)F)c2c1CSc1c(c(C(F)(F)F)nn1C)CS2] |
| (VII′) | (VIIa′) |
| | 1. n = 0, R¹ is F |
| | 2. n = 0, R¹ is OCHF₂ |
| 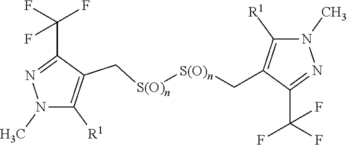 | 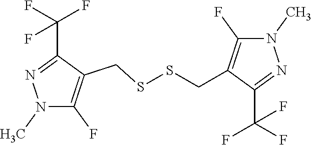 |
| (VIII′) | (VIIIa′) |
| | 1. n = 0, R¹ is F |
| | 2. n = 0, R¹ is OCHF₂ |
| | 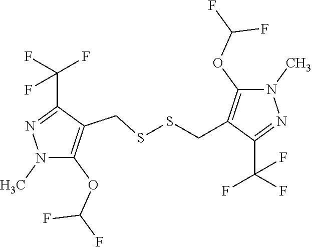 |
| | (VIIIb′) |

According to an embodiment, the present invention provides a compound of formula (II) substantially free from a compound of formula (VI′).

In another embodiment, the present invention provides a compound of formula (II) substantially free from a compound of formula (VIa′).

In another embodiment, the present invention provides a compound of formula (II) substantially free from a compound of formula (VIb′).

In an embodiment, the compound of formula (VI′) is a reaction product produced by the reaction between two molecules of the compound of formula III.

According to an embodiment, the present invention provides a compound of formula (II) substantially free from a compound of formula (VII′).

In another embodiment, the present invention provides a compound of formula (II) substantially free from a compound of formula (VIIa′).

In an embodiment, the compound of formula (VII′) is a reaction product produced by an intermolecular cyclisation of two molecules of the compound of formula (III).

According to an embodiment, the present invention provides a compound of formula (II) substantially free from a compound of formula (VIII′).

In another embodiment, the present invention provides a compound of formula (II) substantially free from a compound of formula (VIIIa′).

In another embodiment, the present invention provides a compound of formula (II) substantially free from a compound of formula (VIIIb′).

In an embodiment, the compound of formula (VIII′) is a reaction product produced by the reaction between two molecules of the compound of formula (III).

According to an aspect of the present invention, there is provided a compound of formula (II);

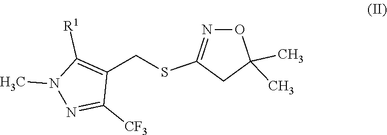

wherein the compound of formula (II) is substantially free of a specific side product which is represented as a compound of formula (V);

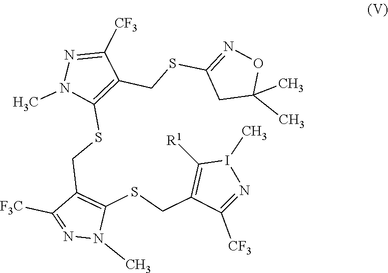

and wherein R¹ is selected from hydrogen, halogen, C₁ to C₁₀ alkyl group, C₃ to C₈ cycloalkyl group, C₃ to C₈ cycloalkyl group, C₁ to C₃ alkyl group or haloalkyl group.

In a preferred embodiment, the compound of formula (II) is substantially free from a compound of formula (V), and wherein R¹ is selected from fluoro or difluoromethoxy group.

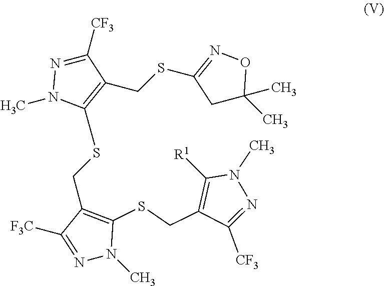

According to an embodiment, there is provided a compound of formula (II) substantially free from a compound of formula (V); wherein R¹ is selected from hydrogen, halogen, C₁ to C₁₀ alkyl group, C₃ to C₈ cycloalkyl group, C₃ to C₈ cycloalkyl group, C₁ to C₃ alkyl group or haloalkyl group.

According to an embodiment, the compound of formula (II) comprises preferably less than 1% (by weight) of the compound of formula (V); and wherein R¹ is selected from hydrogen, halogen, C₁ to C₁₀ alkyl group, C₃ to C₈ cycloalkyl group, C₃ to C₈ cycloalkyl group, C₁ to C₃ alkyl group or haloalkyl group.

According to an embodiment, the compound of formula (II) prepared according to the present invention can further be characterised by physical properties such as bulk density and particle size.

According to another aspect, there is provided a process for preparation of pyroxasulfone of formula (I), the process comprising:
a) preparing a compound of formula (IIa) by condensing a compound of formula (IIIa) or salts thereof, with a compound of formula (IV) in presence of a weak base; wherein reaction is carried out in absence of N,N-dimethyl formamide;

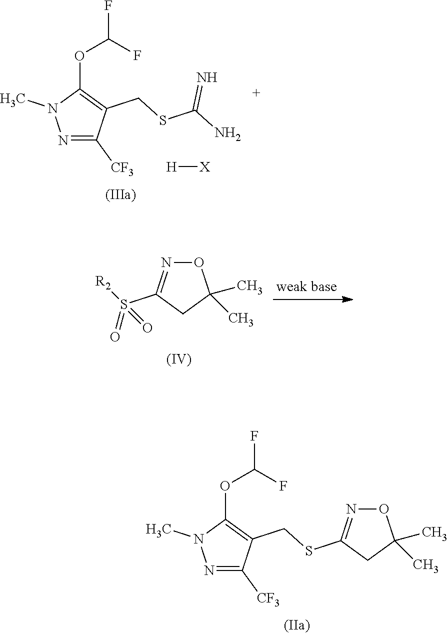

wherein X is halogen and R² is C₁ to C₁₀ alkyl group; and
b) oxidizing the compound of formula (IIa) to obtain pyroxasulfone of formula (I).

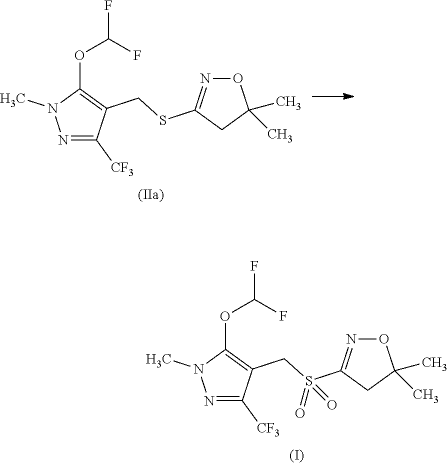

According to an embodiment, the weak base used in step a) of the process for preparation of the compound of formula (IIa) is an inorganic base.

According to an embodiment, the inorganic base is selected from the group comprising potassium carbonate, sodium carbonate, potassium bicarbonate, sodium bicarbonate, or combinations thereof.

According to an embodiment, the weak base is potassium carbonate. According to an embodiment, the weak base used in step a) of the process for preparation of the compound of formula (IIa) is an organic base.

According to an embodiment, the organic base is selected from, but not limited to, one or more of triethylamine, trimethylamine, pyridine, triethylene diamine, N,N-dimethyl pyridine, or combinations thereof.

According to an embodiment, the step a) of the process for preparation of the compound of formula (IIa) is carried out in presence of a solvent.

According to an embodiment, the solvent used is selected from the group comprising methanol, ethanol, isopropyl alcohol, acetone, methyl ethyl ketone, methyl acetate, ethyl acetate, isopropyl ether, petroleum ether, tetrahydrofuran, acetonitrile, benzene, toluene, xylene, dichloromethane, dichloroethane, chloroform, carbon tetrachloride, chlorobenzene, water, or mixtures thereof.

According to an embodiment, the step a) of the process for preparation of the compound of formula (IIa), is carried out at temperature ranging from about 0° C. to about 150° C.

According to an embodiment, the step a) of the process for preparation of the compound of formula (IIa), is carried out at temperature ranging from about 10° C. to about 80° C.

According to an embodiment, the step a) of the process for preparation of the compound of formula (IIa), is carried out at temperature ranging from about 40° C. to about 80° C.

According to an embodiment, the step a) of the process for preparation of the compound of formula (IIa), is carried out at temperature ranging from about 20° C. to about 40° C.

According to an embodiment, in step b) oxidation of the compound of formula (IIa) is carried out in presence of an oxidizing agent.

According to an embodiment, the oxidizing agent used may be selected from organic peroxides such as m-chloroperbenzoic acid, performic acid, peracetic acid; and inorganic peroxides such as hydrogen peroxide, potassium permanganate, sodium periodate, or combinations thereof.

According to an embodiment, the oxidising agent used is hydrogen peroxide.

According to an embodiment in step b), pyroxasulfone compound of formula (I) is prepared in presence of a solvent.

According to an embodiment, the solvent used is selected from the group comprising alcohols, ketones, esters, ethers, nitriles, aromatic or aliphatic hydrocarbons, halogenated aromatic or aliphatic hydrocarbons, water, or mixtures thereof.

According to an embodiment, the solvent used is selected from the group comprising methanol, ethanol, isopropyl alcohol, acetone, methyl ethyl ketone, methyl acetate, ethyl acetate, isopropyl ether, petroleum ether, tetrahydrofuran, acetonitrile, benzene, toluene, xylene, dichloromethane, dichloroethane, chloroform, carbon tetrachloride, chlorobenzene, water, or mixtures thereof.

According to an embodiment, the oxidation in step b) is carried out in presence of a metal catalyst and an acid.

According to an embodiment, the metal catalyst is selected from the group comprising tungsten catalyst, molybdenum catalyst, titanium catalyst, zirconium catalyst, or mixtures thereof.

According to an embodiment, the acid used is an inorganic acid or an organic acid.

The inorganic acid c sulfuric acid and hydrochloric acid; and the organic acid is selected from acetic acid and formic acid.

According to another embodiment, the step b) is carried out at a temperature ranging from about 20° C. to about 100° C.

According to an embodiment, the step b) is carried out at a temperature ranging from about 40° C. to about 80° C.

According to another aspect, there is provided a process for preparation of pyroxasulfone of formula (I), the process comprising:
a) preparing a compound of formula (IIb) by condensing a compound of formula (IIIb) or salts thereof with a compound of formula (IV), wherein reaction is carried out in presence of a base and in absence of N,N-dimethyl formamide;

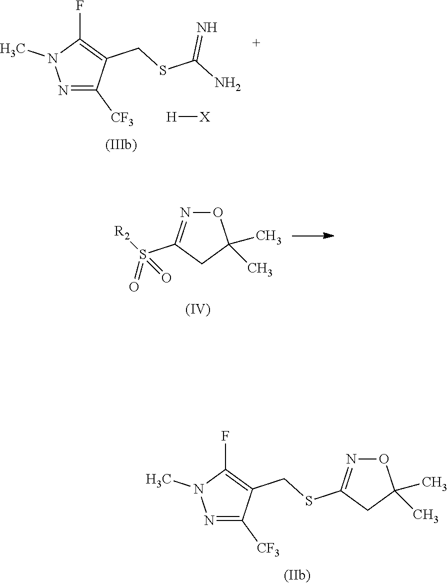

wherein X is halogen and R² is C₁ to C₁₀ alkyl group;
b) converting the compound of formula (IIb) to a compound of formula (IIa);

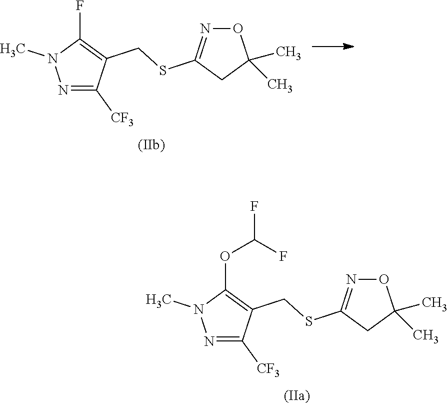

and
c) oxidizing the compound of formula (IIa) to obtain pyroxasulfone of formula (I).

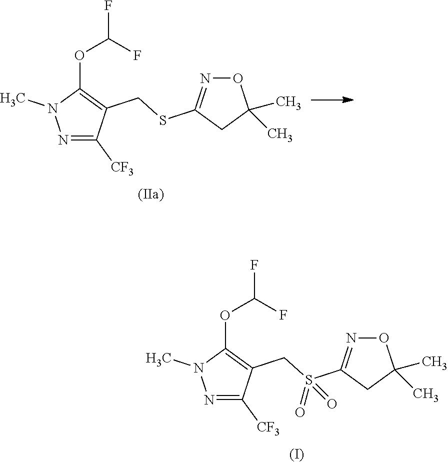

According to an embodiment, the base used in step a) of the process for preparation of the compound of formula (IIb) is an inorganic base.

According to an embodiment, the inorganic base used is selected from, but not limited to, sodium hydroxide, potassium hydroxide, potassium carbonate, sodium carbonate, potassium bicarbonate, sodium bicarbonate, or combinations thereof.

According to an embodiment, the step a) of the process for preparation of the compound of formula (IIb) is carried out in presence of a solvent.

According to an embodiment, the solvent used is selected from the group comprising methanol, ethanol, isopropyl alcohol, acetone, methyl ethyl ketone, methyl acetate, ethyl acetate, isopropyl ether, petroleum ether, tetrahydrofuran, acetonitrile, benzene, toluene, xylene, dichloromethane, dichloroethane, chloroform, carbon tetrachloride, chlorobenzene, water, or mixtures thereof.

According to an embodiment, the step a) of the process for preparation of the compound of formula (IIb) is carried out at temperature ranging from about 0° C. to about 150° C.

According to an embodiment, the step a) of the process for preparation of the compound of formula (IIb) is carried out at temperature ranging from about 10° C. to about 100° C.

According to an embodiment, the step a) of the process for preparation of the compound of formula (IIb) is carried out at temperature ranging from about 20° C. to about 40° C.

According to an embodiment, in step b) of the process, the compound of formula (IIb) is first converted to compound of formula (IIc), followed by converting compound of formula (IIc) to compound of formula (IId), and then finally converting the compound of formula (IId) to compound of formula (IIa).

According to an embodiment, the schematic representation of the reaction of step b) is provided as in Scheme 2.

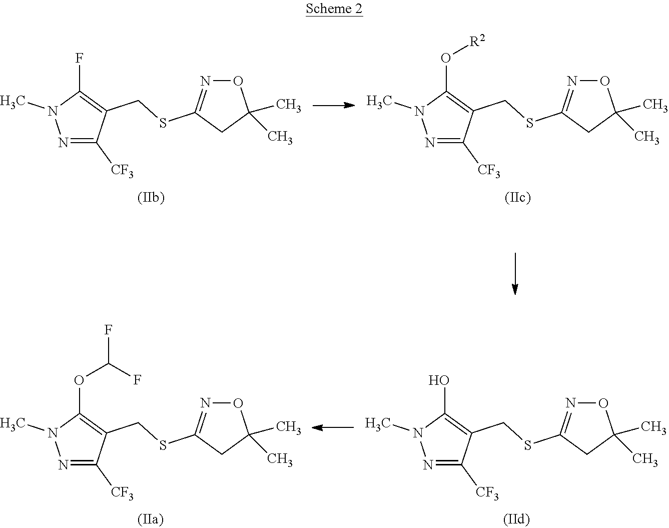

wherein R² is C₁ to C₁₀ alkyl group.

According to another embodiment, in step b) the compound of formula (IIb) is alkoxylated to obtain the compound of formula (IIc), which is then subjected to hydrolysis to get compound of formula (IId), which is further reacted with a fluoromethylating agent to obtain compound of formula (IIa).

According to another embodiment, the compound of formula (IIb) is alkoxylated using an alkoxylating agent, such as sodium methoxide, sodium ethoxide, in presence of an alcohol, such as methanol, to obtain a compound of formula (IIc).

According to another embodiment, the compound of formula (IIc) obtained is converted to the compound of formula (IId) by treatment with an acid selected from an organic acid or an inorganic acid, or mixtures thereof.

According to another embodiment, the organic acid used is selected from the group comprising acetic acid, formic acid, oxalic acid and the like. The inorganic acid used is selected from the group comprising hydrochloric acid, sulfuric acid, nitric acid, phosphoric acid, boric acid, hydrofluoric acid, hydrobromic acid, perchloric acid, hydroiodic acid, or lewis acids such as boron tribromide, boron trichloride, boron trifluoride, or combinations thereof.

According to an embodiment, in step b) reaction is carried out in presence of a fluoromethylating agent, an alkaline reagent and an organic solvent.

According to another embodiment, the step b) is carried out in presence of a fluoromethylating agent such as difluorochloromethane, in presence of an alkaline reagent and an organic solvent.

According to another embodiment, the alkaline reagent used can be inorganic base and/or organic base. The inorganic base is preferably one or more of potassium carbonate, sodium carbonate, potassium bicarbonate, sodium bicarbonate, potassium hydroxide and sodium hydroxide; and the organic base is preferably one or more of triethylamine, trimethylamine, pyridine, triethylene diamine and N,N-dimethyl pyridine.

According to another embodiment, the organic solvent used may be selected from one or more of acetonitrile, N,N-dimethylformamide, tetrahydrofuran, methanol, ethanol, isopropanol, and the like.

According to another embodiment, the step c) is carried out at temperature ranging from about 0° C. to about 150° C.

According to another embodiment, the step c) is carried out at temperature ranging from about 20° C. to about 100° C.

According to another embodiment, the step c) is carried out at temperature ranging from about 40° C. to about 80° C.

According to an embodiment, in step c), the oxidation of compound of formula (IIa) is carried out in presence of an oxidizing agent in an organic solvent to obtain the pyroxasulfone of formula (I).

According to another embodiment, the oxidizing agent used may be selected from organic peroxides such as m-chloroperbenzoic acid, performic acid, peracetic acid; and inorganic peroxides such as hydrogen peroxide, potassium permanganate, sodium periodate.

According to an embodiment, the oxidation is carried out in presence of a metal catalyst and an acid.

According to an embodiment, the metal catalyst is selected from the group comprising tungsten catalyst, molybdenum catalyst, titanium catalyst, zirconium catalyst, or mixtures thereof.

According to another embodiment, the acid used is an inorganic acid or organic acid. The inorganic acid comprises sulfuric acid or hydrochloric acid; and the organic acid comprises acetic acid or formic acid.

According to an embodiment, the step b) is carried out at a temperature ranging from about 40° C. to about 80° C.

According to an embodiment, the present invention provides pyroxasulfone of formula (I) substantially free from impurities. It has been observed that impurities formed during the preparation of the compound of formula (II) undergoes further chemical reaction such as substitution and oxidation reactions during the preparation of pyroxasulfone of formula (I), resulting in multiple complex and structurally similar molecules which are difficult to separate due to their similar physical properties.

According to an embodiment, the present invention provides pyroxasulfone of formula (I) substantially free from unreacted synthetic intermediates, reagents, solvents, organic and/or inorganic products of side reactions, organic and/or inorganic salts, and/or other undesired materials.

In another embodiment, the following table discloses the list of probable organic and/or inorganic products of side reactions, or other undesired compounds, or impurities that are formed during the preparation of pyroxasulfone of formula (I).

| | Undesired products of side reactions in |
|---|---|
| Markush Structure | pyroxasulfone of formula (I) |
| 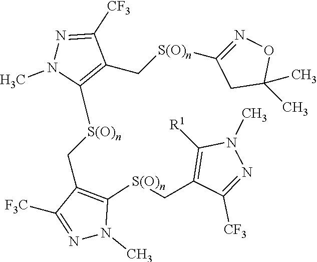 | 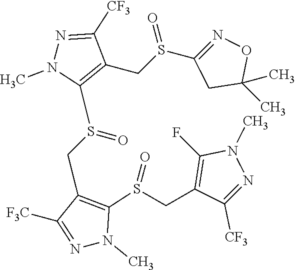 |
| (V′) | (V′c) |
| | 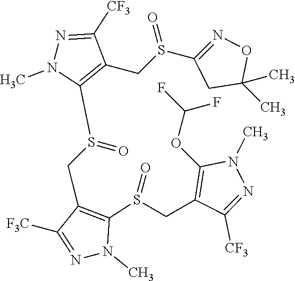 |
| | (V′d) |
| | Other compound formed are sulfone |
| | compounds |
| | 1. n = 2, R¹ is F |
| | 2. n = 2, R¹ is OCHF₂ |
| 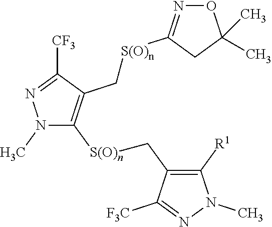 | 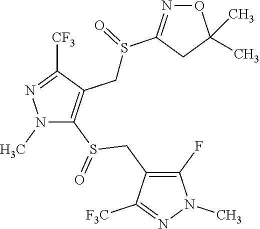 |
| (VI′) | (VIc′) |
| | 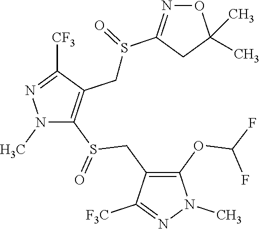 [SMILES: Cn1nc(C(F)(F)F)c(CS(=O)c2c(CS(=O)C3=NOC(C)(C)C3)c(C(F)(F)F)nn2C)c1OC(F)F] |
| | (VId′) |
| | Other compound formed are sulfone |
| | compounds: |
| | 1. n = 2, R¹ is F |
| | 2. n = 2, R¹ is OCHF₂ |
| 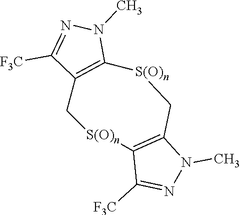 | 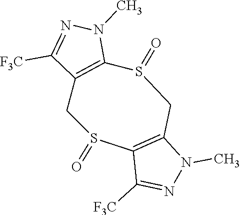 [SMILES: Cn1nc(C(F)(F)F)c2c1CS(=O)c1c(c(C(F)(F)F)nn1C)CS2=O] |
| (VII′) | (VIIb′) |
| | Other compound formed are sulfone |
| | compounds: |
| | 1. n = 2 |
| 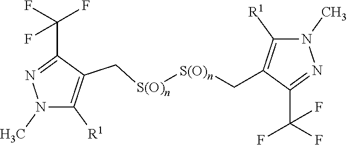 | 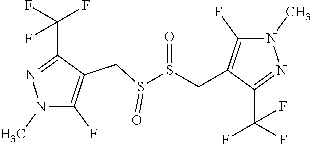 |
| (VIII′) | (VIIIc′) |
| | 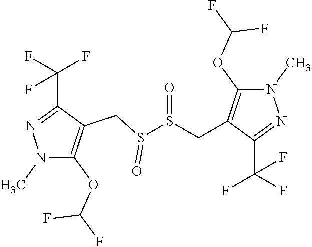 |
| | (VIIId′) |
| | Other compound formed are sulfone |
| | compounds: |
| | 1. n = 2, R¹ is F |
| | 2. n = 2, R¹ is OCHF₂ |

According to another aspect, the present invention provides pyroxasulfone of formula (I)

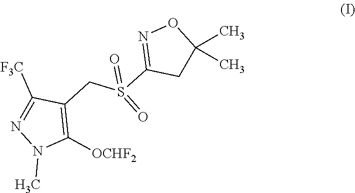 [SMILES: Cn1nc(C(F)(F)F)c(CS(=O)(=O)C2=NOC(C)(C)C2)c1OC(F)F]

wherein pyroxasulfone of formula (I) is substantially free from a compound of formula (V);

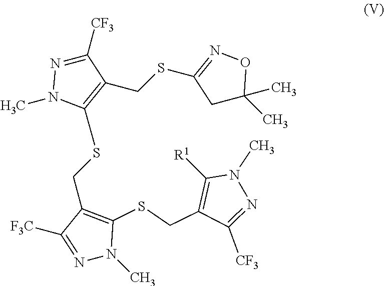

wherein R¹ is selected from hydrogen, halogen, C₁ to C₁₀ alkyl group, C₃ to C₈ cycloalkyl group, C₃ to C₈ cycloalkyl group, C₁ to C₃ alkyl group or haloalkyl group.

According to an embodiment, there is provided a compound of formula (I) substantially free from a compound of formula (V).

According to an embodiment, there is provided a compound of formula (I) comprising less than 1% (by weight) of the compound of formula (V).

According to an embodiment, there is provided a compound of formula (I) comprising less than 1% w/w of the compound of formula (V).

According to an embodiment, there is provided a compound of formula (I) substantially free from a compound of formula (V); wherein R¹ is F.

According to an embodiment, the present invention provides a compound of formula (I) substantially free from a compound of formula (VI′).

In an embodiment, the compound of formula (VI′) is a reaction product produced by the reaction between two molecules of the compound of formula (III).

According to an embodiment, the present invention provides a compound of formula (I) substantially free from a compound of formula (VII′).

In an embodiment, the compound of formula (VII′) is a reaction product produced by an intermolecular cyclisation of two molecules of the compound of formula (III).

According to an embodiment, the present invention provides a compound of formula (I) substantially free from a compound of formula (VIII′).

In an embodiment, the compound of formula (VIII′) is a reaction product produced by the reaction between two molecules of the compound of formula (III).

According to an embodiment, there is provided a process for preparation of a compound of formula (IIb), the process comprising:
condensing a compound of formula (IIIb) or salts thereof, with a compound of formula (IV); wherein reaction is carried out in absence of N,N-dimethyl formamide;

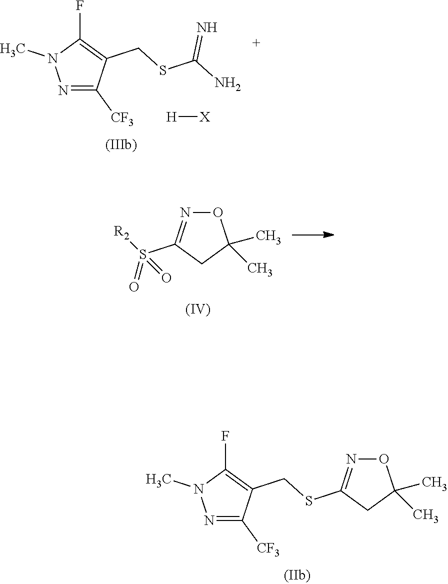

wherein X is halogen and R² is C₁ to C₁₀ alkyl group.

In an embodiment, the compounds of formula (IIIb) and (IV) can be prepared by any method known in the art.

According to an embodiment, the process for preparation of the compound of formula (IIb) is carried out in presence of a base.

According to an embodiment, the base used is selected from, but not limited to, an organic base or an inorganic base.

According to an embodiment, the base used is an inorganic base.

According to an embodiment, the inorganic base used is selected from, but not limited to, sodium hydroxide, potassium hydroxide, potassium carbonate, sodium carbonate, potassium bicarbonate, sodium bicarbonate, or combinations thereof.

According to another embodiment, the base used is an organic base selected from, but not limited to, one or more of triethylamine, trimethylamine, pyridine, triethylene diamine, N,N-dimethyl pyridine, or combinations thereof.

According to an embodiment, the process for preparation of compound of formula (IIb) is carried out in presence of a solvent.

According to an embodiment, the solvent used is selected from, but not limited to alcohol, ketone, ester, ether, nitrile, aromatic or aliphatic hydrocarbon, halogenated aromatic or aliphatic hydrocarbon, water, or mixtures thereof.

According to an embodiment, the solvent used is selected from methanol, ethanol, isopropyl alcohol, acetone, methyl ethyl ketone, methyl acetate, ethyl acetate, isopropyl ether, petroleum ether, tetrahydrofuran, acetonitrile, benzene, toluene, xylene, dichloromethane, dichloroethane, chloroform, carbon tetrachloride, chlorobenzene, water, or mixtures thereof.

According to an embodiment, the process for preparation of compound of formula (IIb) is carried out at temperature ranging from about 0° C. to about 150° C.

According to an embodiment, in the process for preparation of the compound of formula (II), the reaction is carried out at temperature ranging from about 10° C. to about 100° C.

According to an embodiment, in the process for preparation of the compound of formula (II), the reaction is carried out at temperature ranging from about 20° C. to about 40° C.

According to an aspect of the present invention, there is provided a compound of formula (IIb);

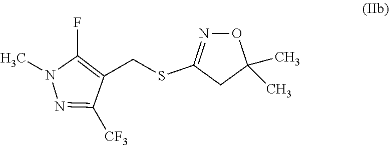

wherein the compound of formula (IIb) is substantially free from a compound of formula (Va);

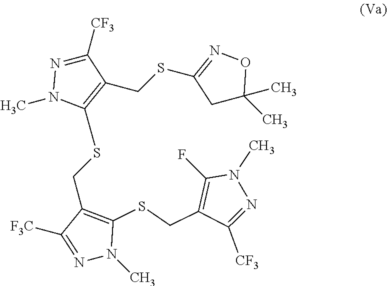

According to an embodiment, there is provided a compound of formula (IIb) substantially free from a compound of formula (Va).

According to an embodiment, there is provided a compound of formula (IIb) comprising less than 1% (by weight) of the compound of formula (Va).

According to an embodiment, there is provided a compound of formula (IIb) comprising less than 1% w/w of the compound of formula (Va).

According to an embodiment, the present invention provides pyroxasulfone of formula (I) substantially free from a compound of formula (V); and wherein R¹ is selected from fluoro or difluoromethoxy group.

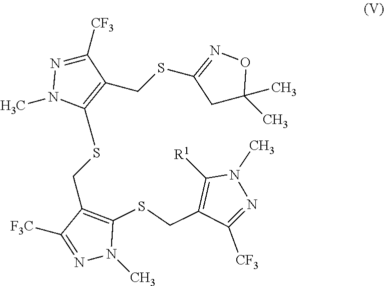

According to an embodiment, pyroxasulfone of formula (I) comprises less than about 1% w/w of the compound of formula (V).

In an embodiment, the present invention provides an agrochemical composition comprising: 3-[5-(difluoromethoxy)-1-methyl-3-(trifluoromethyl)pyrazol-4-ylmethylsulfonyl]-4,5-dihydro-5,5-dimethyl-1,2-oxazole prepared according to the process of the present invention and at least one agrochemically acceptable excipient(s).

In another embodiment, the present invention provides an agrochemical composition comprising a combination of 3-[5-(difluoromethoxy)-1-methyl-3-(trifluoromethyl)pyrazol-4-ylmethyl sulfonyl]-4,5-dihydro-5,5-dimethyl-1,2-oxazole prepared according to the process of the present invention, at least one additional herbicide, and at least one agrochemically acceptable excipient(s).

According to an embodiment, the present invention provides an agrochemical composition comprising: 3-[5-(difluoromethoxy)-1-methyl-3-(trifluoromethyl)pyrazol-4-ylmethylsulfonyl]-4,5-dihydro-5,5-dimethyl-1,2-oxazole prepared according to the present process of the present invention, at least one additional herbicide, and at least one agrochemically acceptable excipient(s).

According to an embodiment of the present invention, the herbicide is selected from the group comprising aliphatic acid herbicides, amide herbicides, aromatic acid herbicides, benzofuran herbicides, benzonitrile herbicides, benzophenone herbicides, benzothiadiazinone herbicides, benzyl ether herbicides, botanical herbicides, carbamate herbicides, cyclohexanedione oxime herbicides, dinitroaniline herbicides, dinitrophenol herbicides, diphenyl ether herbicides, diphenyl heterocyle herbicides, dithiocarbamate herbicides, fumigant herbicides, halogenated alkanoic acid herbicides, imidazolinone herbicides, inorganic herbicides, isoxazole herbicides, isoxazolidinone herbicides, isoxazoline herbicides, N-phenyl heterocycle herbicides, N-phenylimide herbicides, N-phenyloxadiazolone herbicides, N-phenyltriazolinone herbicides, organochlorine herbicides, organophosphorous herbicides, oxirane herbicides, oxyacetamide herbicides, phenol herbicides, phenoxy herbicides, phenylcarboxylic acid herbicides, phenyl ether herbicides, phenylthiourea herbicides, pyrazole herbicides, pyridazine herbicides, pyridazinone herbicides, pyridine herbicides, pyrimidinediamine herbicides, pyrimidinyloxybenzylamine herbicides, quaternary ammonium herbicides, quinone herbicides, alpha-thioacetamide herbicides, thiocarbamate herbicides, thiocarbonate herbicides, triazine herbicides, triazinone herbicides, triazole herbicides, triazolone herbicides, triazolopyrimidine herbicides, triketone herbicides, uracil herbicides, urea herbicides, unclassified herbicides, and combinations thereof.

According to an embodiment of the present invention, the herbicide is selected from the group comprising 2,4-D, bensulfuron-methyl, bentazone, butachlor, carfentrazone, amicarbazone, sulfentrazone, cyhalofop-butyl, haloxyfop-p-methyl, clodinafop-propargyl, clethodim, propanil, imazosulfuron, 2-methyl-4-chlorophenoxyacetic acid (MCPA), mefenacet, napropamide, glufosinate, clomazone, oxadiazon, pendimethalin, pretilachlor, pyrazosulfuron-ethyl, metsulfuron, diflufenican, metribuzin, their derivatives, and mixtures thereof.

According to another embodiment, the agrochemically acceptable excipients are selected from one or more of emulsifier(s), colorant(s), thickener(s)/binder(s), antifreeze agent(s), antifoaming agent(s), antioxidant(s), solvent(s), preservative(s), glidant(s), anticaking agent(s), pH-regulating agent(s), buffering agent(s), formulation aid(s), disintegrant(s), or combinations thereof.

According to yet another embodiment, the agrochemical composition comprising 3-[5-(difluoromethoxy)-1-methyl-3-(trifluoromethyl)pyrazol-4-ylmethylsulfonyl]-4,5-dihydro-5,5-dimethyl-1,2-oxazole prepared according to the process of the present invention and at least one agrochemically acceptable excipient, may be present in a form of a solid formulation or a liquid formulation.

According to yet another embodiment, the agrochemical composition comprising 3-[5-(difluoromethoxy)-1-methyl-3-(trifluoromethyl)pyrazol-4-ylmethylsulfonyl]-4,5-dihydro-5,5-dimethyl-1,2-oxazole prepared according to the process of the present invention and at least one agrochemically acceptable excipient, may be present in a form of a tank-mix or a pre-formulated (pre-mix) formulation.

In another embodiment, the present invention provides use of the agrochemical composition for controlling weeds, wherein the agrochemical composition comprises 3-[5-(difluoromethoxy)-1-methyl-3-(trifluoromethyl)pyrazol-4-ylmethylsulfonyl]-4,5-dihydro-5,5-dimethyl-1,2-oxazole prepared according to the process of the present invention and at least one agrochemically acceptable excipient.

In another embodiment, the present invention provides use of the agrochemical composition as a herbicide, wherein the agrochemical composition comprises 3-[5-(difluoromethoxy)-1-methyl-3-(trifluoromethyl)pyrazol-4-ylmethylsulfonyl]-4,5-dihydro-5,5-dimethyl-1,2-oxazole prepared according to the process of the present invention and at least one agrochemically acceptable excipient.

In another aspect, the present invention provides a method for controlling weeds, said method comprising applying to the plants or locus thereof an agrochemical composition comprising 3-[5-(difluoromethoxy)-1-methyl-3-(trifluoromethyl)pyrazol-4-ylmethylsulfonyl]-4,5-dihydro-5,5-dimethyl-1,2-oxazole prepared according to the process of the present invention and at least one agrochemically acceptable excipient.

In another embodiment, the composition of the present invention is used to control weeds in various agricultural crops.

### Advantages of the Present Invention

1. The present invention discloses a process for preparation of pyroxasulfone and its intermediate of formula (II) in the absence of a hazardous solvent like N, N-dimethyl formamide;
2. The process of the present invention is economical and simple to handle at industrial scale;
3. The process of the present invention is environment friendly; and
4. The present invention discloses a process for preparation of pyroxasulfone and its intermediate of formula (II) having high yield and high purity.

In view of the above, it will be seen that the several advantages of the invention are achieved, and other advantageous results attained. Although the present invention has been disclosed in full, it will be understood that numerous additional modifications and variations could be made thereto without departing from the scope of the invention. The embodiments may be combined together for better understanding of the invention, without departing from the scope of the invention.

In another embodiment, alternative or multiple embodiments of the invention disclosed herein are not to be construed as limitations. Each embodiment can be referred to and claimed individually or in any combination with other embodiments of the invention. One or more embodiments of the invention can be included in, or deleted from, the invention for reasons of convenience and/or patentability.

It will be understood that the specification and examples are illustrative but not limitative of the present invention and that other embodiments within the spirit and scope of the invention will suggest themselves to those skilled in the art. Other embodiments can be practiced that are also within the scope of the present invention. The following examples illustrate the invention, but by no means intend to limit the scope of the claims.

### Examples

### Method Used: High Performance Liquid Chromatography (HPLC)

a) Column—Zorbax SB C-8 [250 millimeter (mm)×4.6 mm, 5 microns (μ)]
b) Mobile Phase—0.1% Ortho phosphoric acid (OPA): Acetonitrile, 90:10
c) Flow—1.0 milliliter (ml)/minute
d) UV-Wavelength—220 nanometer (nm)
e) Injection volume—5 microliter (L)
f) Column temperature—40° C.
g) Run time—30 minutes

### Example 1: Preparation of 3-({[5-fluoro-1-methyl-3-(trifluoromethyl)-1H-pyrazol-4-yl]methyl}sulfanyl)-5,5-dimethyl-4,5-dihydro-1,2-oxazole [Compound of Formula (IIb)]

To a stirred solution of 5-fluoro-1-methyl-3-(trifluoromethyl)-1H-pyrazol-4-yl]methyl carbamimidothioate hydrochloride salt (178.3 g) in ethanol (555.2 g) was added water (18 g) at 25-30° C. followed by addition of 3-(ethylsulfonyl)-5,5-dimethyl-4,5-dihydro-1,2-oxazole (114.19 g). To this mixture potassium carbonate (210.4 g) was added portion-wise in 2-3 hours. The reaction mixture was stirred at 25-30° C. under nitrogen atmosphere for 10-12 hours. After completion of the reaction, ethanol was distilled out under reduced pressure and the organic layer was separated, the aqueous layer was washed with ethyl acetate. The combined organic layer was washed with 5% sulfuric acid followed by washing with brine solution. The organic layer was concentrated under reduced pressure to obtain 184 g of product as yellow oil. (Yield: 96.8%, Purity by HPLC: 86.8% a/a, impurity (compound Va): 0.39%, FIG. 1).

### Example 2: Preparation of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole [Compound of Formula (IIa)]

To a stirred solution of [5-(difluoromethoxy)-1-methyl-3-(trifluoromethyl)-1H-pyrazol-4-yl]methyl carbamimidothioate hydrochloride salt (237.7 g) in ethanol (507 ml) was added water (644 ml) at 25-30° C. followed by addition of 3-(ethylsulfonyl)-5,5-dimethyl-4,5-dihydro-1,2-oxazole (129.5 g). To this mixture potassium carbonate (251 g) was added portion-wise in 2-3 hours. The reaction mixture was stirred at 25-30° C. under nitrogen pressure for 10-12 hours. After completion of reaction, ethanol was distilled out under reduced pressure and the organic layer was separated, the aqueous layer was washed with ethyl acetate. The combined organic layer was washed with 5% sulfuric acid followed by brine solution. The organic layer was concentrated under reduced pressure to obtain 242.1 g of product as yellow oil (Yield: 96.5%, Purity by HPLC: 85.73% a/a).

### Example 3: Preparation of 3-({[5-fluoro-1-methyl-3-(trifluoromethyl)-1H-pyrazol-4-yl]methyl}sulfanyl)-5,5-dimethyl-4,5-dihydro-1,2-oxazole [Compound of Formula (IIb)]

To a stirred solution of 5-fluoro-1-methyl-3-(trifluoromethyl)-1H-pyrazol-4-yl]methyl carbamimidothioate hydrochloride salt (43.75 g) in ethanol (136.2 g) was added water (202 g) at 25-30° C. followed by addition of 5,5-dimethyl-3-(methylsulfonyl)-4,5-dihydro-1,2-oxazole (26 g). To this mixture potassium carbonate (51.65 g) was added portion-wise in 2-3 hours. The reaction mixture was stirred at 25-30° C. under nitrogen pressure for 10-12 hours. After completion of reaction, ethanol was distilled out under reduced pressure and the organic layer was separated, the aqueous layer washed with ethyl acetate. The combined organic layer was washed with 5% sulfuric acid followed by brine solution. The organic layer was concentrated under reduced pressure to obtain 41.39 g of product as yellow oil. (Yield: 90%, Purity by HPLC: 87.7% a/a, impurity (compound Va): 0.56%).

### Example 4: Preparation of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole [Compound of Formula (IIa)]

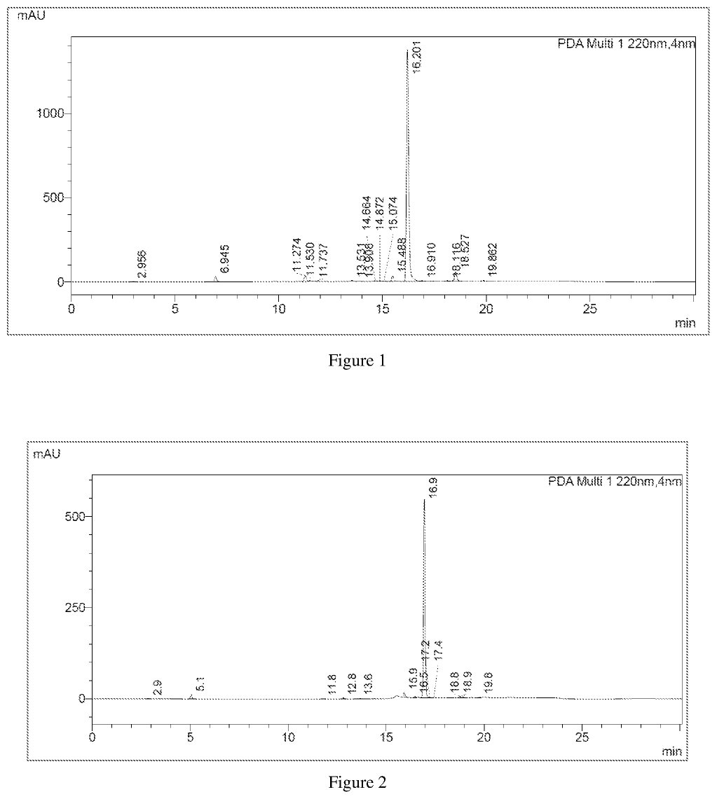

To a stirred solution of [5-(difluoromethoxy)-1-methyl-3-(trifluoromethyl)-1H-pyrazol-4-yl]methyl carbamimidothioate hydrochloride salt (58.44 g) in methanol (118.5 ml) was added water (170 ml) at 25-30° C. followed by addition of 3-(methylsulfonyl)-5,5-dimethyl-4,5-dihydro-1,2-oxazole (29.78 g). To this mixture potassium carbonate flakes (59.25 g) was added portion-wise in 2-3 hours. The reaction mixture was stirred at 25-30° C. under nitrogen atmosphere for 10-12 hours. After completion of reaction, methanol was distilled out under reduced pressure and the organic layer was separated, the aqueous layer was washed with ethyl acetate. The combined organic layer was washed with 5% sulfuric acid followed by brine solution. The organic layer was concentrated under reduced pressure to obtain product as yellow oil (Yield: 92.85%, Purity by HPLC: 91.55% a/a, impurity (compound V): not detected, FIG. 2).

### Example 5: Preparation of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole [Compound of Formula (IIa)]

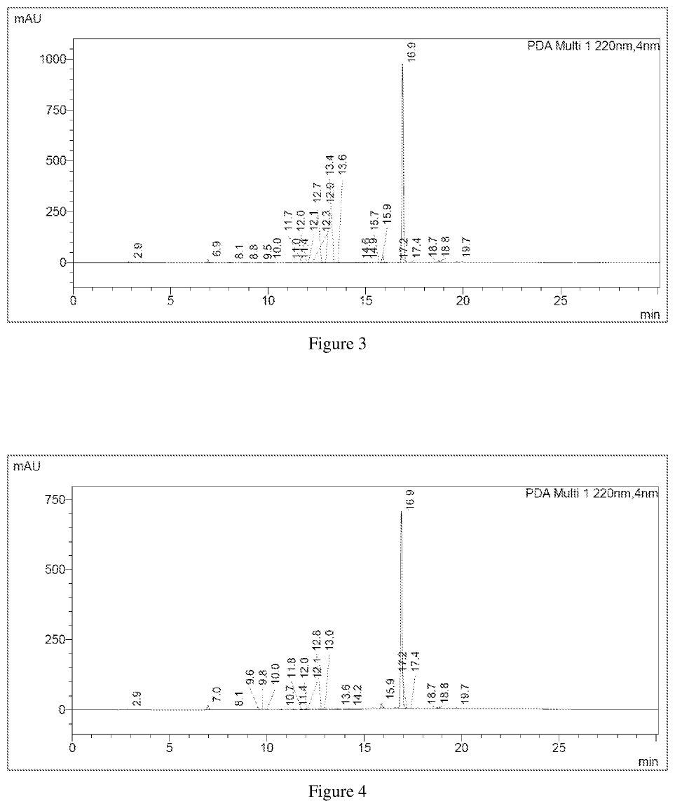

To a stirred solution of [5-(difluoromethoxy)-1-methyl-3-(trifluoromethyl)-1H-pyrazol-4-yl]methyl carbamimidothioate hydrochloride salt (29.22 g) in ethanol (59.22 ml) was added water (85 ml) at 25-30° C. followed by addition of 3-(methylsulfonyl)-5,5-dimethyl-4,5-dihydro-1,2-oxazole (14.88 g). To this mixture sodium carbonate (22.7 g) was added portion-wise in 2-3 hours. The reaction mixture was stirred at 25-30° C. under nitrogen atmosphere for 10-12 hours. After completion of reaction, ethanol was distilled out under reduced pressure and the organic layer was separated, the aqueous layer was washed with ethyl acetate. The combined organic layer was washed with 5% sulfuric acid followed by brine solution. The organic layer was concentrated under reduced pressure to obtain product as yellow oil (Yield: 93.32%, Purity by HPLC: 90.73% a/a, impurity (compound V): not detected, FIG. 3).

### Example 6: Preparation of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole [Compound of Formula (IIa)]

To a stirred solution of [5-(difluoromethoxy)-1-methyl-3-(trifluoromethyl)-1H-pyrazol-4-yl]methyl carbamimidothioate hydrochloride salt (18.9 g) in ethanol (37 ml) was added water (53 ml) at 25-30° C. followed by addition of 3-(methylsulfonyl)-5,5-dimethyl-4,5-dihydro-1,2-oxazole (9.22 g). To this mixture potassium carbonate (18.3 g) was added portion-wise in 2-3 hours. The reaction mixture was stirred at 25-30° C. under nitrogen atmosphere for 10-12 hours. After completion of reaction, methanol was distilled out under reduced pressure and the organic layer was separated, the aqueous layer was washed with ethyl acetate. The combined organic layer was washed with 5% sulfuric acid followed by brine solution. The organic layer was concentrated under reduced pressure to obtain product as yellow oil (Yield: 93%, Purity by HPLC: 92.60% a/a, impurity (compound V): not detected, FIG. 4).

### Example 7: Preparation of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole [Compound of Formula (IIa)]

To a stirred solution of [5-(difluoromethoxy)-1-methyl-3-(trifluoromethyl)-1H-pyrazol-4-yl]methyl carbamimidothioate hydrochloride salt (29.22 g) in ethanol (60 ml) was added water (85 ml) at 25-30° C. followed by addition of 3-(methylsulfonyl)-5,5-dimethyl-4,5-dihydro-1,2-oxazole (14.89 g). To this mixture triethylamine (21.69 g) was added dropwise in 2-3 hours. The reaction mixture was stirred at 25-30° C. under nitrogen atmosphere for 10-12 hours. After completion of reaction, methanol was distilled out under reduced pressure and the organic layer was separated, the aqueous layer was washed with ethyl acetate. The combined organic layer was washed with 5% sulfuric acid followed by brine solution. The organic layer was concentrated under reduced pressure to obtain product as yellow oil (Yield: 86.50%, Purity by HPLC: 92.21% a/a).

### Example 8: Preparation of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole [Compound of Formula (IIa)]

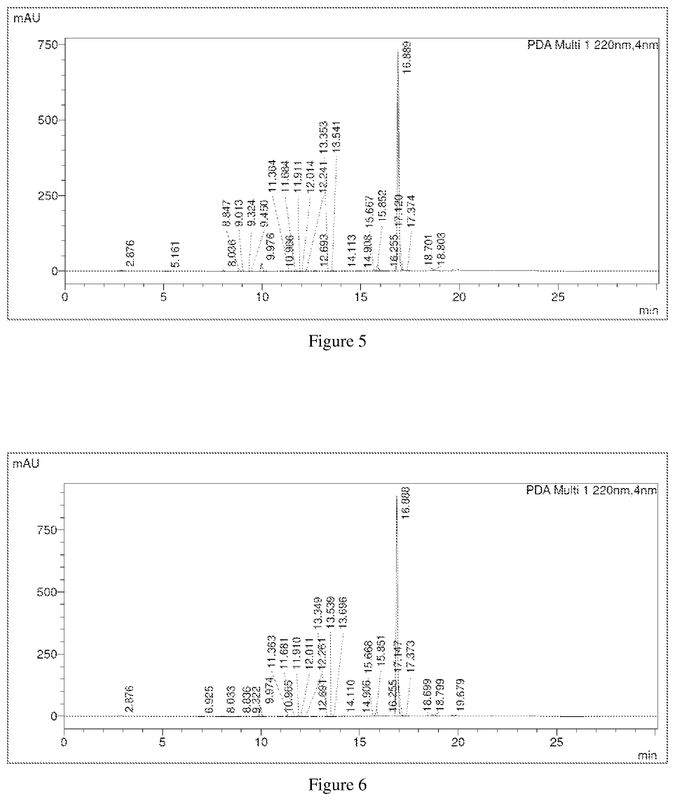

To a stirred solution of [5-(difluoromethoxy)-1-methyl-3-(trifluoromethyl)-1H-pyrazol-4-yl]methyl carbamimidothioate hydrochloride salt (34.74 g) in acetonitrile (63 ml) was added water (101 ml) at 25-30° C. followed by addition of 3-(methylsulfonyl)-5,5-dimethyl-4,5-dihydro-1,2-oxazole (17.7 g). To this mixture potassium carbonate (35.23 g) was added portion-wise in 2-3 hours. The reaction mixture was stirred at 25-30° C. under nitrogen atmosphere for 10-12 hours. After completion of reaction, methanol was distilled out under reduced pressure and the organic layer was separated, the aqueous layer was washed with ethyl acetate. The combined organic layer was washed with 5% sulfuric acid followed by brine solution. The organic layer was concentrated under reduced pressure to product as yellow oil (Yield: 90.46%, Purity by HPLC: 89.61% a/a, impurity (compound V): not detected, FIG. 5).

### Example 9: Preparation of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole [Compound of Formula (IIa)]

To a stirred solution of [5-(difluoromethoxy)-1-methyl-3-(trifluoromethyl)-1H-pyrazol-4-yl]methyl carbamimidothioate hydrochloride salt (34.74 g) in dichloromethane (130 ml) was added water (101 ml) at 25-30° C. followed by addition of 3-(methylsulfonyl)-5,5-dimethyl-4,5-dihydro-1,2-oxazole (17.70 g). To this mixture potassium carbonate (35.23 g) was added portion-wise in 2-3 hours. The reaction mixture was stirred at 25-30° C. under nitrogen atmosphere for 10-12 hours. After completion of reaction, methanol was distilled out under reduced pressure and the organic layer was separated, the aqueous layer was washed with ethyl acetate. The combined organic layer was washed with 5% sulfuric acid followed by brine solution. The organic layer was concentrated under reduced pressure to obtain product as yellow oil (Yield: 91.00%, Purity by HPLC: 88.61% a/a, impurity (compound V): not detected, FIG. 6).

### Example 10: Preparation of 3-({[5-methoxy-1-methyl-3-(trifluoromethyl)-1H-pyrazol-4-yl]methyl}sulfanyl)-5,5-dimethyl-4,5-dihydro-1,2-oxazole [Compound of Formula (IIc), Wherein R² is Methyl]

0.177 mol of 30% sodium methoxide was added to the mixture of 0.071 mol of 3-({[5-fluoro-1-methyl-3-(trifluoromethyl)-1H-pyrazol-4-yl]methyl}sulfanyl)-5,5-dimethyl-4,5-dihydro-1,2-oxazole and 6.17 mol of methanol at 25-30° C. under inert atmosphere. After this, the reaction mixture was heated slowly to reflux and maintained for 5-6 hours. After completion of reaction, methanol was recovered under vacuum followed by addition of 15.16 mol of water and 3.57 mol of ethyl acetate. The mixture was stirred, and layers were separated. The ethyl acetate in organic layer was distilled under vacuum to yield 88% of 3-({[5-methoxy-1-methyl-3-(trifluoromethyl)-1H-pyrazol-4-yl]methyl}sulfanyl)-5,5-dimethyl-4,5-dihydro-1,2-oxazole.

### Example 11: Preparation of 4-{[(5,5-dimethyl-4,5-dihydro-1,2-oxazol-3-yl)sulfanyl]methyl}-1-methyl-3-(trifluoromethyl)-1H-pyrazol-5-ol [Compound of Formula (IId)]

1.278 mol of 25% hydrogen bromide in acetic acid was added to 0.156 mol of 3-({[5-methoxy-1-methyl-3-(trifluoromethyl)-1H-pyrazol-4-yl]methyl}sulfanyl)-5,5-dimethyl-4,5-dihydro-1,2-oxazole, and the mixture was stirred at 25-30° C. for 4-5 hours. After completion of the reaction, acetic acid along with excess hydrogen bromide was recovered completely under vacuum and the product was precipitated by addition of 4.86 mol of water. The precipitated product was filtered, washed with 11.11 mol of water and 1.74 mol of hexane and dried to yield 72% of 4-{[(5,5-dimethyl-4,5-dihydro-1,2-oxazol-3-yl)sulfanyl]methyl}-1-methyl-3-(trifluoromethyl)-1H-pyrazol-5-ol.

### Example 12: 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole [Compound of Formula (IIa)]

0.067 mol of sodium hydroxide was added to the mixture of 0.022 mol of 4-{[(5,5-dimethyl-4,5-dihydro-1,2-oxazol-3-yl)sulfanyl]methyl}-1-methyl-3-(trifluoromethyl)-1H-pyrazol-5-ol and 1.33 mol of acetonitrile with stirring at 22-25° C. temperature. The reaction mixture was maintained at this temperature for 1.5 hours. The mixture was then cooled to 5° C. followed by purging of 0.136 mol of freon gas at 5-15° C. within one hour. The mixture was then maintained at 22-25° C. for 3 hours. After completion of the reaction, 0.282 mol of toluene was added to the mixture followed by addition of 3.33 mol of water and 0.024 mol of 30% hydrochloric acid. The organic layer and aqueous layers were separated. The organic layer was washed with brine and the layer was distilled to yield 84% of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole.

### Example 13: Preparation of Pyroxasulfone

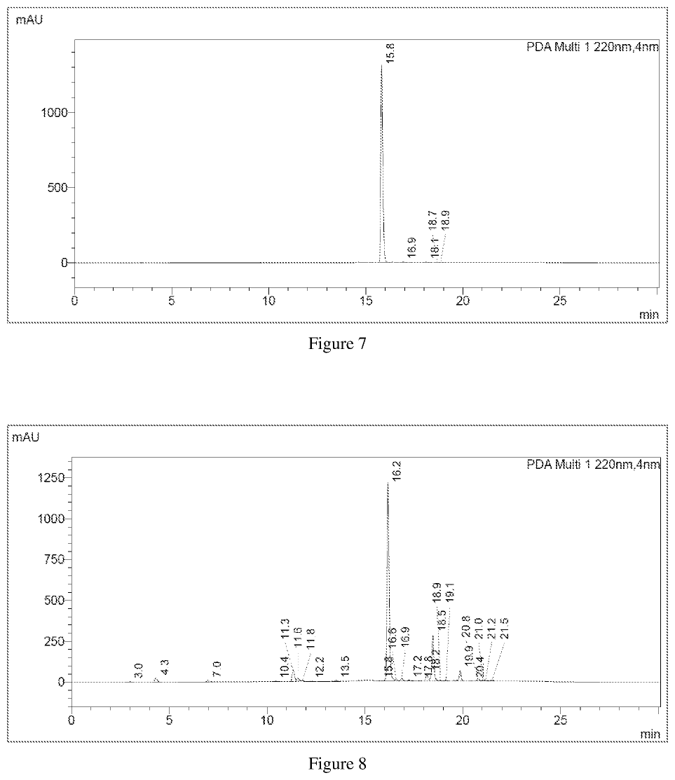

To 31.76 g of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole was added 166 g of acetic acid, 1.28 g of sodium tungstate dihydrate and 22.24 g of 50% hydrogen peroxide. The mixture was stirred at 25-35° C. Then the temperature was increased to 50-55° C. and maintained for another 7 hours. The reaction was monitored by HPLC. After completion of reaction, the mixture was cooled to 25-30° C. and diluted with 48 g of water. The reaction mixture was then cooled to 0° C. and maintained for 1 hour. The product was filtered out washed with water and petroleum ether and dried to yield 30.3 g of Pyroxasulfone technical (Purity by HPLC: 99.46% a/a; impurity (compound V): not detected, FIG. 7).

### Comparative Example 14: Preparation of 3-({[5-fluoro-1-methyl-3-(trifluoromethyl)-1H-pyrazol-4-yl]methyl}sulfanyl)-5,5-dimethyl-4,5-dihydro-1,2-oxazole [Compound of Formula (IIb)]

To a stirred solution of 5-fluoro-1-methyl-3-(trifluoromethyl)-1H-pyrazol-4-yl]methyl carbamimidothioate hydrochloride salt (32.95 g) in ethanol (134 g) was added water (85.3 g) followed by addition of potassium carbonate (18.68 g) at 25-30° C. and stir for 10 minutes to obtain pyrazole solution. In another flask, 3-(ethylsulfonyl)-5,5-dimethyl-4,5-dihydro-1,2-oxazole (21.5 g) in N, N-dimethyl formamide (80.4 g) was taken and potassium carbonate (18.68 g) was added. The mixture was stirred at 25-30° C. for 10 minutes to obtain isoxazole solution. This isoxazole solution was added to the pyrazole solution and stirred at 50° C. for 3-4 hours. The ethanol was distilled off, followed by addition of water and ethyl acetate to the reaction mass. The organic layer was separated and washed with brine solution and concentrated under reduced pressure to obtain 24.79 g of product as yellow oil. (Yield: 71%, Purity by HPLC: 67% a/a, impurity (compound Va): 3.16%, FIG. 8).

### Comparative Example 15: Preparation of 3-({[5-fluoro-1-methyl-3-(trifluoromethyl)-1H-pyrazol-4-yl]methyl}sulfanyl)-5,5-dimethyl-4,5-dihydro-1,2-oxazole (Compound of Formula IIb)

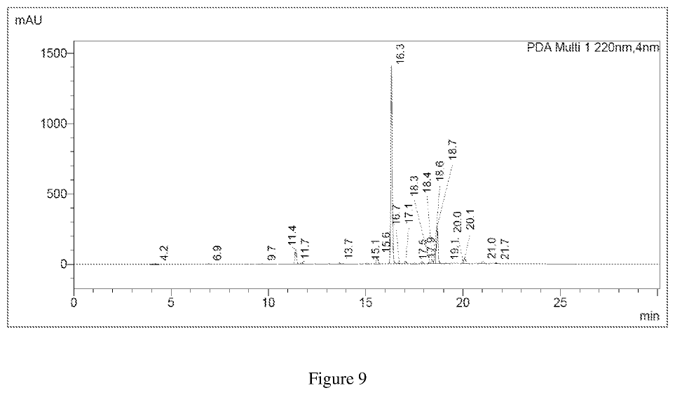

To a stirred solution of 5-fluoro-1-methyl-3-(trifluoromethyl)-1H-pyrazol-4-yl]methyl carbamimidothioate hydrochloride salt (22 g) in ethanol (117.4 g) was added water (75 g) at 25-30° C. followed by addition of mixture of 3-(ethylsulfonyl)-5,5-dimethyl-4,5-dihydro-1,2-oxazole (14.05 g) in N,N-dimethyl formamide (70.71 g). To this mixture potassium carbonate (25.91 g) was added portion-wise in 2-3 hours. The reaction mixture was stirred at 25-30° C. under nitrogen atmosphere for 10-12 hours. After completion of reaction, ethanol was distilled out under reduced pressure and the organic layer was separated, the aqueous washed with ethyl acetate. The combined organic layer was washed with 5% sulfuric acid followed by washing with brine solution. The organic layer was concentrated under reduced pressure to obtain 18.26 g of product as yellow oil. (Yield: 78.4%, Purity by HPLC: 65.24% a/a, impurity (compound Va): 2.03%, FIG. 9).

1. A process for preparation of a compound of formula (II), the process comprising:
condensing a compound of formula (III) or salts thereof with a compound of formula (IV)
in the absence of N,N-dimenthyl formamide; 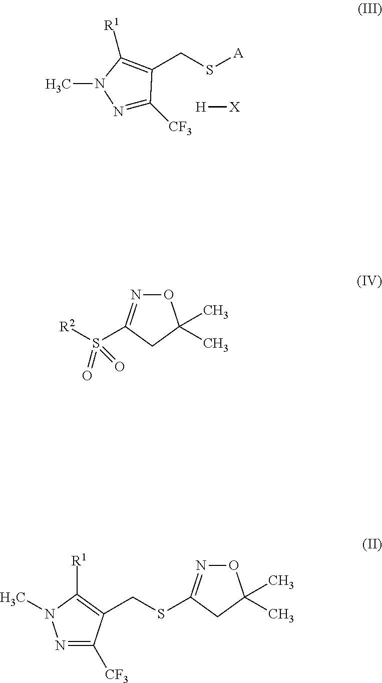
wherein X is a halogen, R² is a C₁ to C₁₀ alkyl group, A is selected from a group consisting of hydrogen, —C(NH)(NH₂), alkali metal, and combinations thereof, and R¹ is selected from the group consisting of hydrogen, halogen, C₁ to C₁₀ alkyl group, C₃ to C₈ cycloalkyl group, C₃ to C₈ cycloalkyl group, C₁ to C₃ alkyl group, haloalkyl group, and combinations thereof.

2. The process as claimed in claim 1, wherein said process is carried out in the presence of a base.

3. The process of claim 2, wherein said base is selected from an organic base or an inorganic base.

4. The process of claim 3, wherein said base is selected from the group consisting of sodium hydroxide, potassium hydroxide, potassium carbonate, sodium carbonate, potassium bicarbonate, sodium bicarbonate, triethylamine, trimethylamine, pyridine, triethylene diamine, N,N-dimethyl pyridine, and combinations thereof.

5. The process of claim 1, wherein said process is carried out in the presence of a solvent.

6. The process of claim 5, wherein said solvent is selected from the group consisting of alcohol selected from methanol, ethanol, isopropyl alcohol; ketone selected from acetone, methyl ethyl ketone; ester selected from methyl acetate, ethyl acetate; ether selected from isopropyl ether, petroleum ether, tetrahydrofuran; nitrile selected from acetonitrile; aromatic or aliphatic hydrocarbon selected from benzene, toluene, xylene, hexane; halogenated aromatic or aliphatic hydrocarbon selected from dichloromethane, dichloroethane, chloroform, carbon tetrachloride, chlorobenzene; water, and mixtures thereof.

7. The process of claim 1, wherein the process is carried out in the presence of a solvent selected from methanol or ethanol.

8. The process of claim 1, wherein the process for preparation of a compound of formula (IIb) comprises:
condensing a compound of formula (IIIb) or salts thereof with a compound of formula (IV)
in the absence of N,N-dimethyl formamide;
wherein X is a halogen and R² is a C₁ to C₁₀ alkyl group.

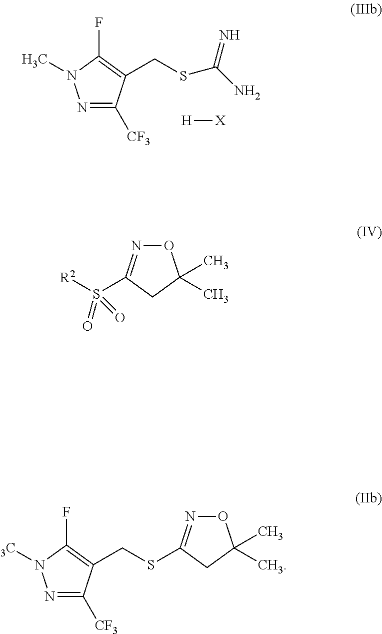

9. A compound of formula (II) substantially free from a compound of formula (V),
wherein R¹ is selected from a fluoro or a difluoromethoxy group.

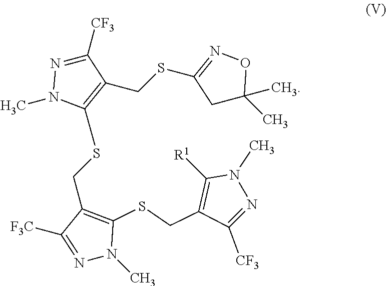

10. A compound of formula (IIb) substantially free from a compound of formula (Va).

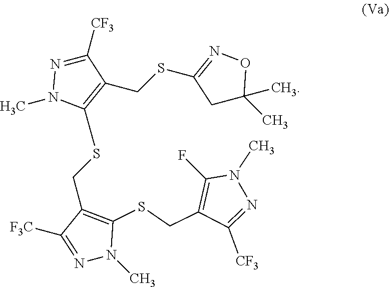 [SMILES: Cn1nc(C(F)(F)F)c(CSc2c(CSc3c(CSC4=NOC(C)(C)C4)c(C(F)(F)F)nn3C)c(C(F)(F)F)nn2C)c1F]

11. The compound of claim 10, wherein the compound of formula (IIb) comprises less than about 1% w/w of the compound of formula (Va).

12. A process for preparation of pyroxasulfone of formula (I), the process comprising:
a) preparing a compound of formula (IIa) by condensing a compound of formula (IIIa) or salts thereof with a compound of formula (IV) in the presence of a base;
wherein the condensing reaction is carried out in absence of N,N-dimethyl formamide; and
b) oxidizing the compound of formula (IIa) to obtain pyroxasulfone of formula (I).

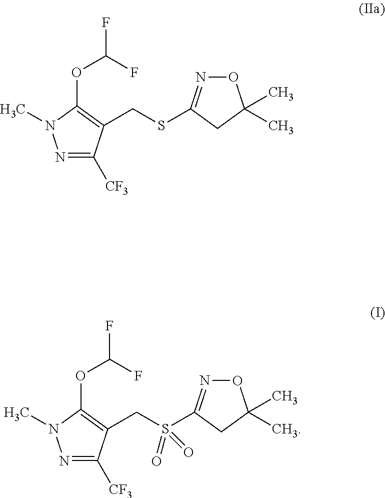

13. The process of claim 12, wherein in said step b) oxidation is carried out in presence of an oxidizing agent.

14. The process of claim 12, wherein said oxidizing agent is selected from the group comprising m-chloroperbenzoic acid, performic acid, peracetic acid, inorganic peroxides comprising hydrogen peroxide, potassium pernanganate, sodium periodate, or combinations thereof.

15. A process for preparation of pyroxasulfone of formula (I), the process comprising:
a) preparing a compound of formula (IIb) by condensing a compound of formula (IIIb) or salts thereof with a compound of formula (IV)
in the absence of N,N-dimethyl formamide; 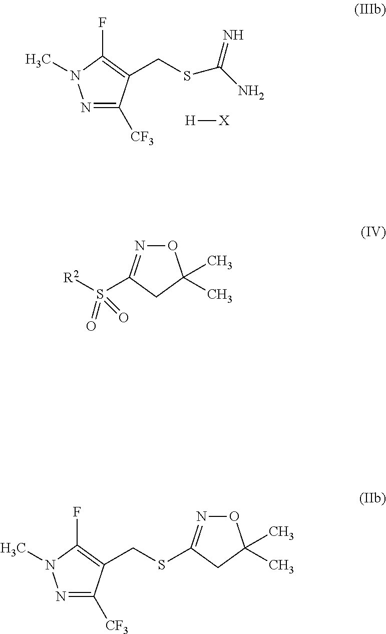
wherein X is a halogen and R² is a C₁ to C₁₀ alkyl group;
b) converting the compound of formula (IIb) to a compound of formula (IIa); and 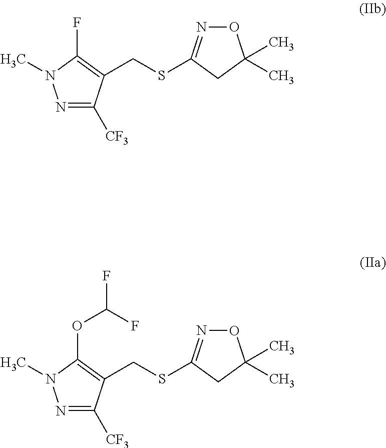
c) oxidizing the compound of formula (IIa) to obtain pyroxasulfone of formula (I).

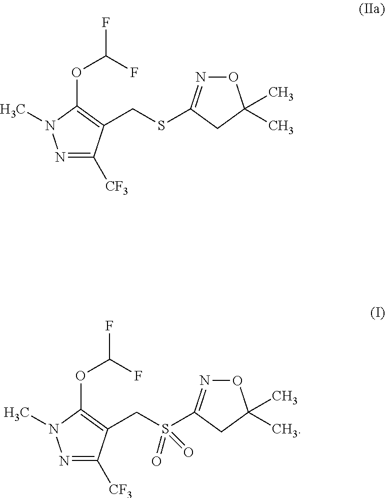

16. The process of claim 15, wherein in step b) reaction is carried out in presence of a fluoromethylating agent, an alkaline reagent and an organic solvent.

17. Pyroxasulfone of formula (I) substantially free from a compound of formula (V);
wherein R¹ is selected from fluoro or difluoromethoxy group.

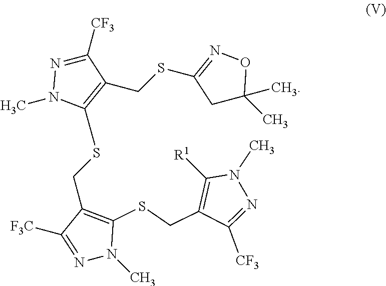

18. The compound of claim 17, wherein pyroxasulfone of formula (I) comprises less than about 1% w/w of the compound of formula (V).
